(19) United States
(12) Patent Application Publication
(10) Pub. No.: US 2019/0376930 A1
(43) Pub. Date: Dec. 12, 2019

(54) Electrochemical Method and Device for Measuring the Different Uncomplexed Forms of Sulphur Dioxide in an Aqueous Liquid Medium

(71) Applicants: Universite de Bordeaux, Bordeaux (FR); Institut Polytechnique de Bordeaux, Talence (FR); Centre National de la Recherche Scientifique, Paris (FR)

(72) Inventors: Stéphane Arbault, Gradignan (FR); Stéphane Reculusa, Pessac (FR); Alexander Kuhn, Guillac (FR); Neso Sojic, Cestas (FR); Pascal Masse, Izon (FR)

(73) Assignees: Universite de Bordeaux, Bordeaux (FR); Institut Polytechnique de Bordeaux, Talence (FR); Centre National de la Recherche Scientifique, Paris (FR)

(21) Appl. No.: 16/487,174

(22) PCT Filed: Feb. 20, 2018
(86) PCT No.: PCT/FR2018/050397

§ 371 Date: Aug. 20, 2019

(30) Foreign Application Priority Data
Feb. 21, 2017 (FR) 1751370

(51) Int. Cl. G01N 27/48; G01N 27/416; G01N 33/00; G01N 33/14

(52) U.S. Cl. CPC G01N 27/48; G01N 33/146; G01N 33/0042; G01N 27/4166

(57) ABSTRACT

The invention relates to an electrochemical method and device for detecting and/or quantifying sulphur dioxide (S(¾) in its various uncomplexed forms (molecular and ionic) in an aqueous or hydroalcoholic liquid food product. It also relates to a method for regenerating an electrode, the active surface of which is composed of gold. The electrochemical method for detecting and/or quantifying sulphur dioxide (SO2) in its uncomplexed (free) forms in an aqueous or hydroalcoholic liquid food product of the invention comprises: a) introducing into the liquid food product a measuring electrode, the active surface of which, in contact with the liquid food product is entirely made of gold, b) measuring the variation of the current produced by the oxidisation of the free SC½, present in the food liquid, during a potential sweep by cyclic voltammetry. In particular, the invention is used for measuring free S(¾).

The invention relates to a method and an electrochemical device for the detection and/or quantification of sulphur dioxide (SO₂) in its different non-complexed forms (molecular and ionic) in an aqueous or hydroalcoholic liquid-food. It also relates to a process for regenerating a measurement electrode of which the active surface is entirely in gold.

In the following of this present document, non-complexed SO₂ will be referred to as the term “free SO₂”.

Owing to its antiseptic, antioxidant and antioxidasic properties, SO₂ is widely used for the conservation of fruit and in the elaboration of aqueous beverages, such as non-fermented fruit juices or hydroalcoholic beverages, such as sparkling or non-sparkling wines, champagnes, ciders, beers and fermented fruit juices.

The addition of SO₂ in different chemical forms, called sulphiting, in particular takes place throughout the wine elaboration process, from the vinification to the packaging, in order to protect, disinfect and preserve it. During the incorporation of SO₂ in a fermenting must or in a wine, a fraction of it will combine with the sugars, the aldehydes (mainly ethanal) or the ketones present in this medium rich in organic compounds. The non-complexed remaining fraction, called free fraction, is the one which possesses the most interesting properties. The most antiseptic fraction of the free SO₂ is called active SO₂ and chemically corresponds to molecular SO₂. The active SO₂ content is a function of the pH, the temperature, the alcohol content and the concentration in free SO₂. It is of great oenological interest as it reflects the wine's level of protection against the oxidation and the contamination by spoilage microorganisms. Thus, the winemaker has to know the free SO₂ concentration throughout the wine's lifecycle in order to adjust the SO₂ quantities. Free SO₂ is the sulphur dioxide present in the following forms: H₂SO₃, HSO₃⁻ and SO₃²⁻, of which the equilibrium is a function of the pH and the temperature.

H₂SO₃⇄H⁺+HSO₃⁻. with pK1=1.9

HSO₃⁻⇄H⁺+SO₃²⁻. with pK2=7.2

In wine, of which the pH is typically comprised between 3 and 4, the H₂SO₃ and HSO₃ forms are the majority.

Currently, two methods are used by winemakers to dose the free SO₂ present in must or wine.

The first method is called the “Ripper method” and is described in the international compendium of analysis methods—OIV-MA-AS323-04B.

This commonly used method consists in an iodometric dosage of the free SO₂ in an acid medium and of the combined SO₂ after alkaline hydrolysis on wine samples.

The second method is the method called “Franz Paul” and is described in the international compendium of analysis methods—OIV-MA-AS323-04A.

In this method, the H₂SO₃ formed by acidification of the medium is driven by a current of air or nitrogen; it is fixed and oxidised into sulphuric acid (H₂SO₄) by bubbling in a neutral dilute solution of hydrogen peroxide.

The H₂SO₄ thus formed is dosed by a titrated solution of sodium hydroxide. The free SO₂ is extracted from the wine by cold extraction (about 10° C.) and the combined SO₂ by warm extraction.

As can be seen, these analysis methods can only be implemented by a specialised laboratory, and outside of the vineyard site.

These methods use indirect measurements that require the collection of a samples and the addition of reagents. Therefore, their protocols are complex to implement.

Thus, there is a need for a method and a device for quick and direct detection and/or quantification of the free SO₂, which can be implemented by a non-specialist in vineyards.

For this purpose, the invention proposes an electrochemical method for the detection and/or quantification of the free SO₂ in an aqueous or hydroalcoholic liquid-food, characterised in that it comprises:
a) the introduction in the liquid-food of:
   a measurement electrode of which the active surface in contact with the liquid-food is entirely in gold,
   a reference electrode,
   a counter-electrode, and
b) the measurement of the variation of the current produced by the oxidisation of the free SO₂ present in the liquid-food, during potential scanning by cyclic voltammetry.

According to an embodiment of the method of the invention, at step b), the potential scanning is performed:
by using an Ag/AgCl electrode as reference electrode,
between −1 V and +2 V, preferably between +0.1 V and +1.9 V, and more preferably between +0.1 V et +1.5 V,
at potential scanning speeds comprised between 1 mV·s⁻¹ and 10,000 mV·s⁻¹, preferably comprised between 10 mV·s⁻¹ and 1,000 mV·s⁻¹, more preferably comprised between 10 mV·s⁻¹ and 100 mV·s⁻¹.

Preferably, the liquid-food is wine.

According to a preferred embodiment of the method of the invention, the measurement electrode has a Rₜₕₑₒ ratio between its developed surface area (A_{dev}) and its apparent surface area (Aₐₚₚ) greater than or equal to 3, preferably comprised between 3 and 100, and more preferably comprised between 3 and 20.

According to another embodiment of the method of the invention, the method further comprises, prior to step b), a step b1) of potential scanning(s), during a cyclic voltammetry, performed:
by using an Ag/AgCl electrode as reference electrode,
between +0.1 V et +1.5 V,
at potential scanning speeds comprised between 1 mV·s⁻¹ and 10,000 mV·s⁻¹, preferably comprised between 10 mV·s⁻¹ and 1,000 mV·s⁻¹, more preferably comprised between 10 mV·s⁻¹ and 100 mV·s⁻¹, without measurement of the current variation, enabling the oxidisation and then the reduction of the active surface entirely in gold of the measurement electrode in contact with the liquid-food.

According to an embodiment of the method of the invention, this method further comprises:
prior to step b), a step b2) of establishing an abacus connecting the pH value of the liquid-food as a function of the position of the peak of reduction of gold oxides during potential scans, by cyclic voltammetry, performed in the liquid-food at different pH values, using an Ag/AgCl electrode as reference electrode, between +1 V and +2 V, preferably between +0.1 V and +1.9 V, more preferably between +0.1 V and +1.5 V, and at potential scanning speeds comprised between 1 mV·s⁻¹ and 10,000 mV·s⁻¹, preferably comprised between 10 mV·s⁻¹ and 1,000 mV·s⁻¹, more preferably comprised between 10 mV·s⁻¹ and 100 mV·s⁻¹,
the measurement of the temperature T of the liquid-food, during step b), and
the determination of the alcohol titration, ABV, of the liquid-food,

which provides the molecular SO₂ percentage contained in the free SO₂, thanks to the following formula:

molecular SO₂%=100/[10^{(pH-pK1)+}1]

wherein pK₁=1.949+(T−20)×0.0322+(ABV−10)×0.01971

The invention also proposes a method for regenerating a measurement electrode of which the active surface is entirely in gold, characterised in that it comprises:

a) the introduction in a liquid-food of the electrode with an active surface entirely in gold, and

b) the implementation of one or potential scan(s), during a cyclic voltammetry, performed:
by using an Ag/AgCl electrode as reference electrode,
between +0.1 V et +1.5 V,
at potential scanning speeds comprised between 1 mV·s⁻¹ and 10,000 mV·s⁻¹, preferably comprised between 10 mV·s⁻¹ and 1,000 mV·s⁻¹, more preferably comprised between 10 mV·s⁻¹ and 100 mV·s⁻¹, without measurement of the current variation, enabling the oxidisation and then the reduction of the part of active surface in gold of the measurement electrode in contact with the liquid-food.

The invention also proposes an electrochemical device for the detection and/or quantification of the free SO₂ in an aqueous or hydroalcoholic liquid-food, characterised in that it comprises:
a reference electrode,
a counter-electrode,
a measurement electrode of which the active surface is entirely in gold,

and characterised in that the measurement electrode has a Rₜₕₑₒ ratio between its developed surface area (A_{dev}) and its apparent surface area (Aₐₚₚ) greater than or equal to 3, preferably comprised between 3 and 100, and more preferably comprised between 3 and 20.

According to an embodiment of the device of the invention, the measurement electrode is an electrode comprising a support covered with a porous sheath in gold, this sheath having a thickness and pore sizes such that the measurement electrode has a Rₜₕₑₒ ratio between its developed surface area (A_{dev}) and its apparent surface area (Aₐₚₚ) greater than or equal to 3, preferably comprised between 3 and 100, more preferably comprised between 3 and 20. This porous sheath forms the active surface of the measurement electrode.

The size of the pores and the thickness of the porous sheath are measured by scanning electron microscope (SEM).

According to another embodiment of the device of the invention, the measurement electrode is an electrode comprising a support covered with a rough layer in gold, constituted of gold crystallites of nano to micrometric dimensions such that the measurement electrode has a Rₜₕₑₒ ratio between its developed surface area (A_{dev}) and its apparent surface area (Aₐₚₚ) greater than or equal to 3, preferably comprised between 3 and 100, and more preferably comprised between 3 and 20. This rough layer in gold forms the active surface of the measurement electrode.

The invention will be better understood and other characteristics and advantages of the invention will appear more clearly on reading the explanatory description that follows and which is made with reference to the drawings, wherein:

FIG. 1 is a diagram of a device according to the invention,

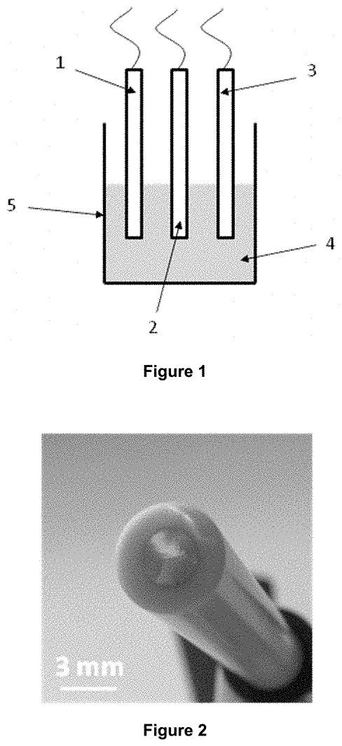

FIG. 2 represents a first measurement electrode, called “planar disc”, used for the detection and/or quantification of the invention,

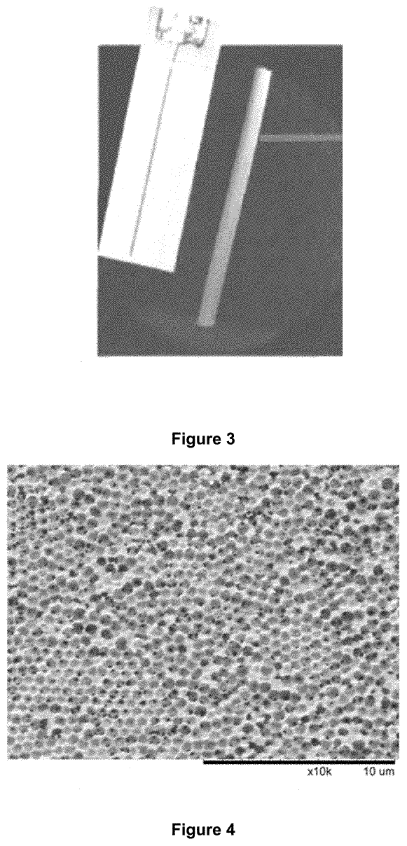

FIG. 3 shows a second measurement electrode, called “porous microstructured”, in gold and used in the device and method for the detection and/or quantification of the invention,

FIG. 4 represents a photograph taken with a scanning electron microscope (SEM), magnified 1000 times, of the surface of the electrode represented in FIG. 3,

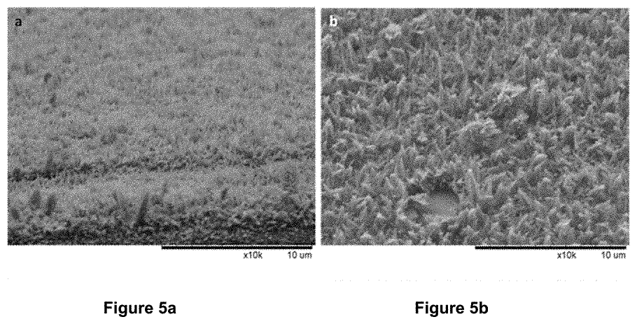

FIGS. 5a and 5b show images taken with the SEM, magnified 1000 times, of the surface of a cylindrical measurement electrode called “rough microstructured” of which the surface is covered with gold crystallites;

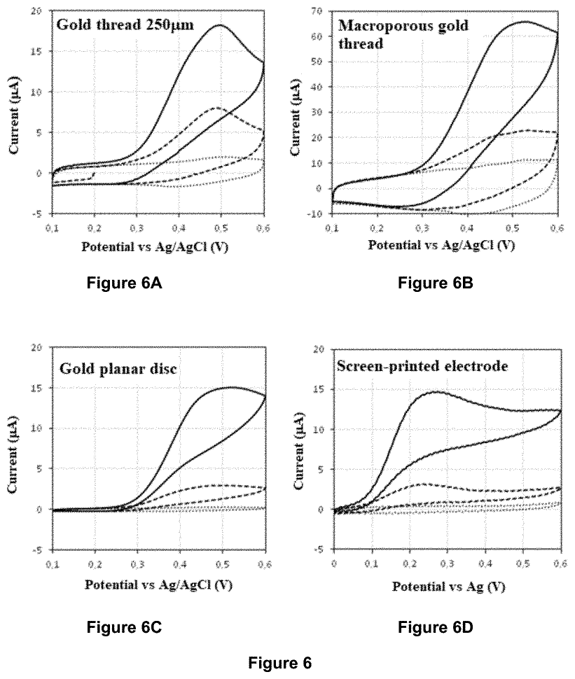

FIG. 6 shows the cyclic voltammetry curves of model hydroalcoholic solutions with addition 0.25 and 125 mg·L⁻¹ of free SO₂ (respectively dotted curve, dashed curve and solid-line curve) obtained by using, as measurement electrode:
a measurement electrode constituted of a gold cylinder of 25 mm in length and of a diameter of 250 μm (FIG. 6A),
the microstructured porous electrode shown in FIGS. 3 and 4 (FIG. 6B),
the microstructured rough electrode represented in FIG. 5 (FIG. 6B),
the planar disc electrode shown in FIG. 2 (FIG. 6C), and
a measurement electrode in gold of a device marketed by the company Dropsens that further comprises a reference electrode in Ag, and a counter-electrode, all of these electrodes being deposited by screen printing in a support in a polymer material (FIG. 6D),

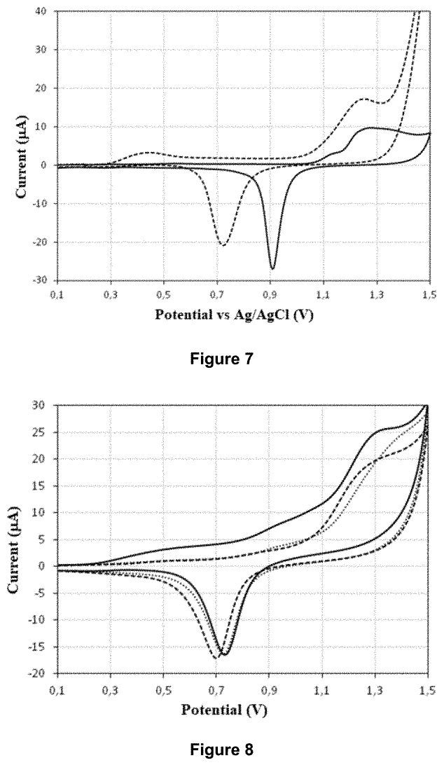

FIG. 7 shows the cyclic voltammetry curves obtained in a solution of sulphuric acid 0.1 M (solid-line curve) and in a model hydroalcoholic solution containing 25 mg·L⁻¹ of free SO₂ (dashed curve),

FIG. 8 shows the cyclic voltammetry curves obtained with a measurement electrode in gold and of the planar disc type in a white wine (solid-line curve), a rosé wine (dotted curve) and in a red wine (dashed curve),

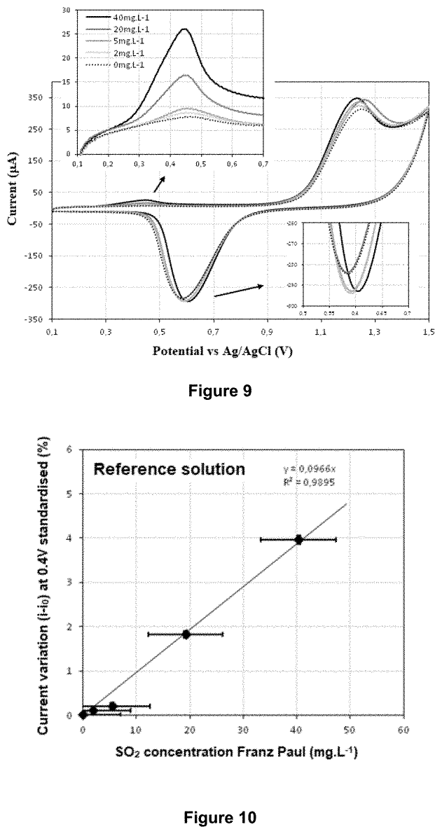

FIG. 9 shows the cyclic voltammetry curves recorded in model hydroalcoholic solutions containing variable contents of free SO₂ using as a measurement electrode a porous microstructured electrode in gold,

FIG. 10 shows the regression curve of the variation of the standardised current measured at +0.4 V as a function of the concentration of free SO₂ in a model hydroalcoholic solution,

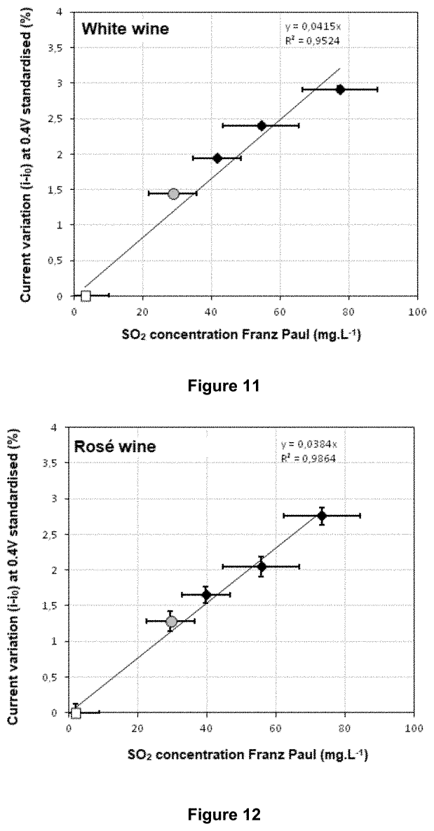

FIG. 11 shows the regression curves of the variation of the standardised current measured at +0.4 V as a function of the concentration of SO₂ measured by the Franz Paul method in:
a white wine (Bordeaux, Sauvignon Roche Mazet, 2014) (grey round symbol), for the same white wine to which ethanal has been added (white square symbol), and
the same white wine with dosed additions of SO₂ of 20 mg·L⁻¹, 40 mg·L⁻¹ and 70 mg·L⁻¹ (black diamond symbols),

FIG. 12 shows the regression curves of the variation of the standardised current measured at +0.4 V as a function of the concentration of SO₂ measured by the Franz Paul method for:
a rosé wine (Cabernet d'Anjou, Plessis Duval, 2015) (grey round symbol),
the same rosé wine to which ethanal has been added (white square symbol), and
the same rosé wine with dosed additions of SO₂ of 20 mg·L⁻¹, 40 mg·L⁻¹ and 70 mg·L⁻¹ (black diamond symbols),

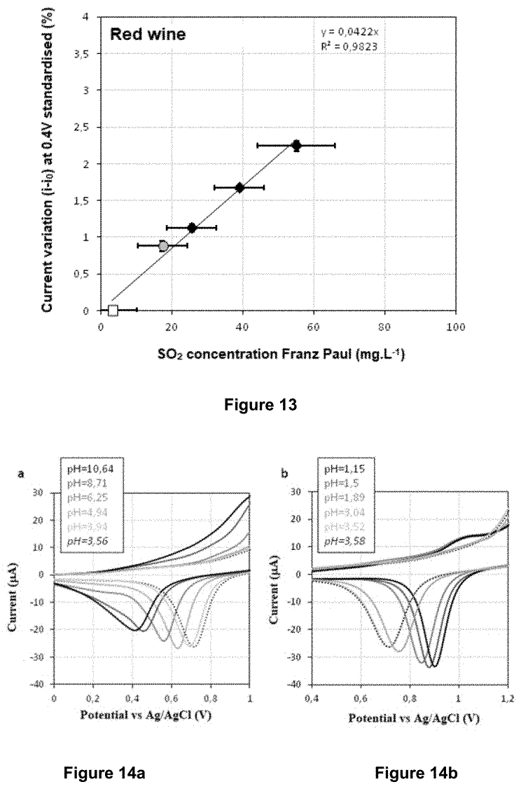

FIG. 13 shows the regression curves of the variation of the standardised current measured at +0.4 V as a function of the concentration of SO₂ measured by the Franz Paul method for:
a red wine (Morgon, Club des sommeliers, 2014) (grey round symbol),
the same red wine to which ethanal has been added (white square symbol), and
the same red wine with dosed additions of SO₂ of 20 mg·L⁻¹, 40 mg·L⁻¹ and 70 mg·L⁻¹ (black diamond symbols),

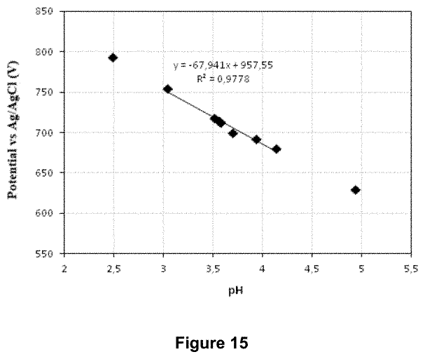

FIG. 14a shows the cyclic voltammetry curves obtained by using as measurement electrode the planar disc electrode shown in FIG. 2, and recorded in a white wine (dotted curve) and by varying the pH with additions of soda (solid-line curves),

FIG. 14b shows the cyclic voltammetry curves obtained by using as measurement electrode, the planar disc electrode shown in FIG. 2 and recorded in a white wine (dotted curve) and by varying the pH by adding sulphuric acid (solid-line curves), and

FIG. 15 shows the variation of the position of the peak of reduction of gold oxides as a function of the pH for a white wine when the planar disc electrode shown in FIG. 2 is used as measurement electrode.

The invention proposes a method for the detection and/or quantification of the free SO₂ in an aqueous or hydroalcoholic liquid-food based on an electrochemical measurement performed by cyclic voltammetry wherein an electrode, of which the active surface is entirely in gold, is used as measurement electrode.

In the present invention, the term “active surface” refers to the surface of the layer intended to be electrically connected to a potentiostat and that reacts with the sulphites.

In the present invention, the term “active surface entirely in gold” means that the active surface is solely constituted of gold with a purity greater than 95%, preferably greater than 99%.

In the present invention, the terms “aqueous liquid-food” or “hydroalcoholic” means, for aqueous liquid-foods, fruit juice in particular and, for hydroalcoholic liquid-foods, sparkling or non-sparkling wine, champagne, cider, fruit-based alcohols and beer.

The quantification and detection of the free SO₂ are performed in a three-electrode electrochemical cell schematically schematized in FIG. 1. It comprises three electrodes: a measurement electrode, noted 1 in FIG. 1, a reference electrode, noted 2 in FIG. 1, and a counter-electrode, noted 3 in FIG. 1, which are immersed in a liquid-food, noted 4 in FIG. 1, contained in a container, noted 5 in FIG. 1, of which the free SO₂ content is to be determined. These three electrodes are connected to a potentiostat (not shown) that can transmit the measured data to a computer.

The measurement electrode 1 can be a planar disc type measurement electrode, shown in FIG. 2, that is constituted by the straight section of a gold thread, for example of 3 mm in diameter, coated in an insulating body, for example in Teflon®.

The measurement electrode 1 can also be a gold cylinder, for example with a length of 25 mm and a diameter of 250 μm.

The measurement electrode 1 can also be a measurement electrode in gold deposited by screen printing on a polymer support where, in addition to the measurement electrode in gold, a silver reference electrode and a counter-electrode in gold are also deposited by screen printing on the same support in polymer.

This assembly constitutes a disposable system that can therefore be directly immersed in the sample for the measurement and connected to a potentiostat.

However, advantageously, the measurement electrode 1 will be a porous microstructured electrode, as represented in FIG. 3, which consists of a cylindrical support of which at least the outer surface is in gold, with a length of approximately 25 mm and a diameter of 250 μm, said outer surface being coated with a porous sheath in gold enabling to increase its active surface. This porous sheath forms the active surface of the measurement electrode. According to an embodiment, the cylindrical support can be entirely constituted of gold. However, the cylinder can be formed of a glass or a metal core other than gold, and coated with a layer of gold. This layer of gold forms the active surface of the measurement electrode. Although the support shown in FIG. 3 is cylindrical, it will clearly appear to the person skilled in the art that the support can be a planar support, or even hemispherical.

Porous microstructured electrodes, such as those represented in FIG. 3 can be achieved as described in international application WO 2016/030806: layers of spherical silica particles with controlled sizes are deposited on the surface of the cylinder. The diameter of the deposited silica particles can vary from 50 nm to 5 μm. Then, an electrodeposition of gold through the interstices of the silica particle film is performed. Then, the silica particles are eliminated by a chemical treatment, which enables the elaboration of a sheath having a porous periodic structure in gold, the size of the pores being adjusted by the diameter of the particles. The thickness of this porous sheath can be controlled between the equivalent of the mid-height of a layer of particles up to a height corresponding to 50 layers of particles, i.e. a thickness of 25 nm to several hundreds of μm. Such an electrode thus develops a large specific surface area corresponding to the active surface of the measurement electrode, while maintaining a small dimension. Such a porous microstructured electrode is shown in FIG. 3. FIG. 4 shows the outer surface of this electrode corresponding to the active surface of the measurement electrode.

The measurement electrode can also be a rough microstructured electrode, i.e. comprising a rough surface in gold constituted of a network of gold crystallites, of nano to micrometric dimension (from 100 μm to 1 nm). This rough gold surface forms the active surface of the measurement electrode. Such an electrode is shown in FIGS. 5a and 5b. The rough microstructured surface was obtained by electrodeposition of a gold salt (tetrachloroauric acid) in the presence of lead acetate as described in Plowman B. J., Ippolito S. J., Bansal V., Sabri Y. M., O'Mullane A. P., Bhargava S. K. Chem. Commun., 2009, 33, 5039, on a support that can be cylindrical, planar or spherical, in gold or in a material different from gold but covered by a layer of gold. The duration of the electrodeposition makes it possible to control the size of the gold needles and therefore the Rₜₕₑₒ ratio of the obtained electrode. Thus, the rough microstructured gold electrode shown in FIG. 5a, for which the duration of electrodeposition was of 300 s, has a Rₜₕₑₒ ratio of 4.9 and the rough microstructured gold electrode shown in FIG. 5b, for which the duration of electrodeposition was of 600s, is characterised by a Rₜₕₑₒ ratio of 12.6.

It can be seen that when the specific surface of the measurement electrode in gold was increased, it presented greater sensitivity of detection and/or quantification of the free SO₂ while maintaining a small dimension. This is why, in the electrochemical detection device of the invention, the measurement electrode in gold presenting a structured porous active surface (the porous microstructured electrode) is preferred.

The term “structured or microstructured porous surface” means that the Rₜₕₑₒ ratio between the developed surface area and the apparent surface area is greater than or equal to 3. Preferably, this ratio comprised between 3 and 100, and more particularly between 3 and 20.

The apparent surface area, noted as Aₐₚₚ, is defined as the geometric area of the electrode, i.e. for a planar electrode, the calculation method is simply to multiply the length by the width of the surface of this electrode.

The developed surface area, noted A_{dev}, is defined as the maximum exposed surface area that is able to interact with the surrounding solution. It corresponds to the real surface area and reflects all the possible structures of the surface of the material (porosity, roughness, etc.).

This Rₜₕₑₒ ratio is of about 1 for the planar disc electrode and for the bare cylindrical electrode in gold (without rough or porous microstructuration), used to detect and/or quantify the free SO₂ in the invention.

For the porous microstructured electrode of which the porous sheath is constituted of seven and a half porous layers of gold, with pores of 1170 nm of average diameter measured by SEM, this Rₜₕₑₒ ratio is of about 11.8 and for the porous microstructured electrode, of which the porous sheath is constituted of two and a half layers of gold with pores of 585 nm of average diameter measured by SEM, this Rₜₕₑₒ ratio is of about 3.9.

The measurement electrode 1 can be a porous microstructured electrode obtained by deposition using a method of printing a porous microstructured layer of gold on a support of which the surface can be, for example, in carbon, platinum, silver or gold.

The measurement electrode 1 can also comprise a protective or selective membrane surrounding the active surface entirely in gold.

This membrane can be an exclusion of size or charge (anionic or cationic charge) membrane deposited by different methods such as drop-casting, dip-coating, laminar deposition or electrophoresis.

The method of the invention makes it possible to quantify very precisely the free SO₂ present in a liquid-food thanks to a previously generated calibration curve.

Indeed, the golden electrode features an excellent selectivity for free SO₂ and it is possible to regenerate its active surface directly in the aqueous or hydroalcoholic liquid-food, which makes it possible to extend its life span, while improving the repeatability of the performed measurements.

In the present invention, cyclic voltammetry is used which consists of applying a linear potential scanning between an initial potential and a final potential, and measuring the current variations resulting from the transfer of electrons generated by the oxidation or reduction processes produced during the cycle.

In the invention it consists in applying oxidation potentials, then reduction in return.

This offers the advantage of reducing species created during the oxidation and thus returning to the initial potential, and thus to the initial state of the measurement electrode at the end of each cycle.

This method presents thus two technical advantages with respect to other single-variant methods, such as linear voltammetry:
after several measurement cycles, it is possible to obtain a stable electrochemical response of the electrode, which makes it possible to disregard the variations of the initial surface state of the electrode; and
the measurement, during a cycle, of a peak of current characteristic of the reduction of the gold oxides makes it possible to standardise the currents via a precise calculation of the electrochemically active surface and thus to overcome the behaviour differences related to the nature of the matrix used;

According to the present invention, the potential scanning of the cyclic voltammetry can be performed between −1 V and +2 V, but is preferably performed between +0.1 V and +1.9 V, and more preferably between +0.1 V and +1.5 V. These potential values are those applied using an Ag/AgCl reference electrode, at potential scanning speeds comprised between 1 mV·s⁻¹ and 10,000 mV·s⁻¹, preferably comprised between 10 mV·s⁻¹ and 1,000 mV·s⁻¹, more preferably comprised between 10 mV·s⁻¹ and 100 mV·s⁻¹.

However, according to an advantageous embodiment of the method of the invention, the range of potentials is defined so as to enable, on one hand, to measure the oxidation peak of the free SO₂ and, as will be seen in the examples, on the other hand, to regenerate the measurement electrode during the measurement cycle. Thus preferably, a potential of from +0.1 V to +1.5 V is applied using an Ag/AgCl reference electrode, at potential scanning speeds comprised between 1 mV·s⁻¹ and 10,000 mV·s⁻¹, preferably comprised between 10 mV·s⁻¹ and 1,000 mV·s⁻¹, more preferably comprised between 10 mV·s⁻¹ and 100 mV·s⁻¹.

The current variation produced by the oxidation reaction of the free SO₂ is then measured, except in the case of the regeneration of the electrode.

The invention further proposes an electrochemical device for the detection and/or quantification of the free SO₂ in an aqueous or a hydroalcoholic liquid-food comprising a reference electrode 2, preferably in Ag/AgCl to fix the potential, a counter-electrode 3, preferably in a conducting polymer, such as polycarbonate charged with carbon particles, poly(pyrrole), and a porous or rough microstructured measurement electrode 1, in gold, such as defined above.

However, it is also possible to use, as a reference electrode 2, a normal hydrogen electrode or a KCl- or NaCl-saturated calomel electrode.

In the same way, a counter-electrode 3 in a noble metal (platinum), stainless steel or non-degradable carbon can also be used.

The cyclic voltammetry measurement method is, as is demonstrated in the following examples, the electrochemical method to be applied to detect and/or quantify in a precise, selective and repeatable manner, the free SO₂ in an aqueous or hydroalcoholic liquid-food.

In order to better understand the invention, as well as its advantages, several embodiments provided by way of purely illustrative and non-limiting examples are now going to be described.

In these examples, the reference electrode is an Ag/AgCl electrode, except when the electrode used is a screen-printed electrode, in which case the reference electrode is in Ag. The potential scanning speed is maintained at 50 mV·s⁻¹.

### Example 1: Comparison of the Measurement Electrodes in Model Solutions

Model solutions having a composition close to those of wine but presenting a simpler matrix have been prepared.

The matrix of these model solutions was constituted of water, ethanol, and tartaric acid. The quantity of ethanol was constant and equal to 12% by volume, the concentration of tartaric acid was fixed at 5 g·L⁻¹ and the pH was adjusted at 3.3 by addition of soda at 30%.

The quantity of free SO₂ was controlled in a range of concentrations from 0 mg·L⁻¹ to 250 mg·L⁻¹ by adding a variable quantity of a sulphur dioxide (SO₂) solution of which the titration was quantified by the Franz Paul method.

In these model solutions, the totality of the added SO₂ remains in the form of free SO₂ in the solution because they do not contain any compounds that are able to combine with a part of the added SO₂.

In this example, the electrochemical responses of the model solutions containing an increasing quantity of free SO₂ were studied using different measurement electrodes. For each form of measurement electrode, the measurements are performed with an increasing quantity of free SO₂. These quantities are respectively 0.25 and 125 mg·L⁻¹ of free SO₂.

Thus, have been tested:
a gold electrode constituted of a bare gold thread with a length of 20 mm and a diameter of 250 μm having an apparent surface area of 15.6 mm² and a Rₜₕₑₒ of 0.8: FIG. 6A,
a porous microstructured gold electrode constituted of a gold thread covered in a gold porous sheath having an apparent surface area of 15.6 mm² and a Rₜₕₑₒ=11.4: FIG. 6B,
a planar disc gold electrode with a diameter of 3 mm having an apparent surface area of 7 mm² and a Rₜₕₑₒ of 1.3: FIG. 6C, and
a screen-printed gold electrode having an apparent surface area of 12 mm² and a Rₜₕₑₒ of 1: FIG. 6D.

All these voltammograms show a current increase when the concentration of free SO₂ increases (0.25 et 125 mg·L⁻¹), from +0.1 V (vs Ag/AgCl) for the screen-printed electrode and from +0.3 V (vs Ag/AgCl for the three other types of measurement electrodes used. This reflects the specificity of the electrochemical response to the free SO₂ of the measurement electrode in gold, this species being the only one of which the concentration varies.

The recorded current increases correspond to the oxidisation of the free SO₂. It can be seen that the intensity of the measured currents is higher when the measurement electrode is a porous microstructured electrode in gold. This electrode is thus preferred as it presents a great sensitivity.

The porous microstructured gold electrode tested here is an electrode of which the outer sheath has a thickness of 15 μm and pores with an average diameter of 1170 nm measured by SEM. This outer sheath forms the active surface of the measurement electrode.

The other three electrodes present comparable measured current intensities, despite their different apparent surface areas, but consistent with their value of the Rₜₕₑₒ ratio close to 1.

It can also be seen from FIG. 6 that the position of the maximum current is shifted towards +0.25 V in the case of the screen-printed electrode, because the reference electrode (in this case in Ag) is different from that of the other three systems (reference electrode in Ag/AgCl).

### Example 2: Repeatability of the Measurements and Regeneration of the Measurement Electrode

The repeatability of the electrochemical response of the model hydroalcoholic solutions containing 125 mg·L⁻¹ of free SO₂ was studied using the planar disc electrode in gold without prior electrochemical cleaning thereof.

The obtained voltammograms show a great disparity of the responses, both in terms of the intensity and of the position of the maximum of the oxidation waveform of the free SO₂. This reveals that the state of surface of the electrode is not the same before each experiment because of the adsorption of molecules at the surface of the electrode.

The regeneration of the surface of the electrode, prior to each measurement, is thus necessary.

A standard protocol for the regeneration of the measurement electrode used in laboratories consists of performing cycles in a large range of potentials (between +0.1 V and +1.5 V) in a solution of sulphuric acid (H₂SO₄) with a concentration comprised between 0.1 and 0.5 M, until obtaining a stable signal.

The cyclic voltammogram obtained with the planar disc electrode in gold is characteristic of a gold electrode with the appearance of a waveform at +1.3 V, which corresponds to the oxidisation of the gold surface and an intense peak at +0.9 V in the cathodic branch due to a reduction of the gold oxides previously generated.

Model hydroalcoholic solutions having a pH of 3.3 were used to confirm or refute that the measurement electrodes used in the device and the method of the invention could be regenerated directly in the medium containing the free SO₂, the acidity thereof being thus sufficient.

Thus, cycles in a potential range between +0.1 V and +1.5V in the model solution containing 25 mg·L⁻¹ of free SO₂ were performed.

The obtained cyclic voltammetry curves are shown in FIG. 7. They show the oxidisation waveform of the free SO₂ at +0.45 V, as well as the prime peak of the gold oxidisation at around +1.25 V and the well-defined peak of reduction of the gold oxides at +0.75 V.

The layer of oxides created during the anodic regime can lead to a decrease in the measured current and a progressive loss of sensitivity of the electrode.

The electrochemical reduction of the gold oxides during the cathodic regime then makes it possible to eliminate this inhibiting layer, by cleaning and reactivating the surface of the electrode.

It was thus demonstrated that it is possible to regenerate the measurement electrode directly in the measurement medium, i.e. in the aqueous or hydroalcoholic liquid-food, which is a definite advantage.

Such a regeneration method, during which the variation of the current intensity is not measured, but only an obtained stable signal is observed, is an object of the invention.

### Example 3: Determination of the Scanning Potential Window During Cyclic Voltammetry

To optimise the scanning potential window for cyclic voltammetry in the method according to the invention, the potential window was submitted to variations, while maintaining a lower threshold equal to +0.1 V, i.e. the foot of the waveform of the oxidisation of the free SO₂, and scanning beyond the first gold oxidisation peak (approximately +1.2 V) towards more positive potentials of +1.5 V to +1.9 V.

From +1.4 V, the signal increases strongly due to the oxidation of water into dioxygen. There is no influence of the upper potential limit on the oxidation peak of the free SO₂.

However, to decrease the measurement time, the upper limit of the potential window is preferably of +1.5 V.

The potential window has also been modified by acting on the lower potential limit, from −0.4 V to +0.1 V.

A waveform appears in the cathodic regime around −0.2 V, which corresponds to the reduction of the free SO₂ into sulphur.

Because of its great affinity with gold, the sulphur thus formed is adsorbed on the surface of the measurement electrode. The response of the free SO₂ is thus modified.

Furthermore, a slight shift of the oxidisation peak of the sulphites towards lower potentials and a decrease of the peak's intensity have been observed.

On the other hand, the plot of the oxidisation current variation measured at the potential of +0.4 V as a function of the concentration in free SO₂ presents a regression coefficient very close to 1 when the cyclic voltammetry measurement is performed between +0.1 V and +1.5 V, whereas the linearity of the response to low concentrations of free SO₂ (<15 mg·L⁻¹) is lost for the cyclic voltammetry measurement performed between −0.4 V and +1.5 V.

Thus, in the method of the invention, the cyclic voltammetry measurement must be performed between +0.1 V and +1.9V, and more preferably between +0.1 V and +1.5 V, this window indeed makes it possible to regenerate the electrode while having a specific response to the free SO₂.

To confirm this point, FIG. 8 shows cyclic voltammograms recorded using a planar disc electrode in red wine, rosé wine and white wine, at potentials from +0.1 V to +1.5V.

This voltammogram shows a gold oxidisation peak at around +1.25 V and a very symmetrical peak of the reduction of gold oxides at around +0.75 V.

The surface of the measurement electrode can therefore be directly regenerated in different wines, without having to perform this regeneration operation in a solution of sulphuric acid.

Repeatability measures were performed by recording the electrochemical response of six samples per wine.

All the voltammetry curves present an inflexion at around +0.5 V corresponding to the oxidisation current peak of the free SO₂ on the gold electrode.

For all the wines, a good repeatability was observed in the slope of the peak of oxidisation of the free SO₂ (between +0.1 V and +0.4 V), the deviation between the measurements being more significant beyond +0.4 V.

The standard deviations in % obtained on the oxidisation currents measured at +0.4 V are satisfactory for three types of analysed wines.

| | White | Rosé | Red |
|---|---|---|---|
| Current | 1.46 ± 0.2 | 0.80 ± 0.04 | 0.57 ± 0.05 |
| at +0.4 V (μA) | (14%) | (5%) | (9%) |

The oxidisation current is more important for white wine and of the same order of magnitude for rosé and red, which tends to show that the concentration in free SO₂ is greater in white wine than in the other two wines.

This is confirmed by the dosage of the free SO₂ contained in these wines, performed by a device implementing the Ripper method.

| | White | Rosé | Red |
|---|---|---|---|
| Free SO₂ Ripper | 55 ± 11 | 11 ± 7 | 14 ± 7 |
| (mg · L⁻¹) | (20%) | (63%) | (50%) |

### Example 4: Establishment of a Standardised Calibration Curve in the Model Solution

In this example, cyclic voltammograms were recorded between +0.1 V and +1.5 V with the porous microstructured gold measurement electrode.

This porous microstructured measurement electrode is constituted of a gold thread with a length of 25 mm and a diameter of 250 μm, presenting a porous surface layer, with a thickness of 2.7 μm and an average pores diameter, measured by SEM, of 585 nm.

The samples are hydroalcoholic model solutions containing variable quantities of free SO₂ and controlled by adding a variable quantity of a solution of SO₂, of which the titration is quantified by the Franz Paul method.

FIG. 9 shows the obtained cyclic voltammetry curves.

As for the planar disc gold electrode, a quantitative increase of the current with the concentration of free SO₂ is recorded at +0.2 V, with a maximum at approximately +0.45 V, which corresponds to the oxidisation of the free SO₂.

The fact of applying to the sample a linear scanning in a wide range of potentials (between +0.1 V and +1.5 V), enables both to measure the oxidisation peak of the free SO₂ but also to regenerate the microstructured measurement electrode directly in the sample with the appearance of a waveform around +1.2 V, which corresponds to the oxidisation of the gold surface of the electrode, and an intense peak around +0.6 V in the cathodic branch, due to the reduction of the generated gold oxides.

It can be seen from the voltammograms shown in FIG. 9, that the porous microstructured electrode presents a greater sensitivity for the dosage of the free SO₂, the currents varying between −350 pA and +350 pA, whereas with a planar disc electrode, they vary between −50 pA and +50 pA.

Moreover, the regression curve of the variation of the current measured at +0.4 V as a function of the concentration of free SO₂ in a solution containing from 0 to 40 mg·L⁻¹ of added SO₂ was plotted in FIG. 10.

The variations of the oxidisation currents measured at +0.4 V are standardised by the height of the reduction peak of the gold oxides observed in each cyclic voltammogram so as to take into account the in situ real active surface of the electrode during the measurements.

As can be seen in FIG. 10, when the value of the oxidisation current measured at the potential of +0.4 V is plotted as a function of the concentration in free SO₂ measured by the Franz Paul method, a regression coefficient very close to 1 is obtained, which once again reflects the fact that the recorded electrochemical response is, in fact, very specific to the free SO₂, which is the only species of which the concentration varies linearly.

It is also seen from FIG. 9 that there is a shift of the position of the peak of reduction of the gold oxides towards more positive potentials with the increase of the concentration in free SO₂.

The potential position of the peak of reduction of gold oxides depending on the pH, this deviation confirms the acidification of the solution by the increase of the quantity of SO₂. It is therefore possible to link the position of this peak to the pH of the solution, and to establish a calibration curve (abacus) that will make it possible to determine the pH of the liquid-food based on the position of the peak of reduction of gold oxides in this liquid-food, during a cyclic voltammetry.

Thus, the electrochemical device and the electrochemical method of the invention also make it possible to measure the pH of the liquid-food, without having to add a new electrode.

This is an additional advantage of the electrochemical device and the electrochemical method of the invention.

### Example 5: Establishment of Standardised Calibration Curves in Different Wines: White Wine, Rosé Wine and Red Wine

In the same way, the regression curves of the variation of standardised current measured at +0.4 V as a function of the concentration of free SO₂ measured by the Franz Paul method were plotted for a white wine, a rosé wine and a red wine (FIGS. 11, 12, 13 respectively).

The measurement electrode used is the same as that used for example 4 (porous microstructured measurement electrode constituted of a gold thread with a length of 25 mm and a diameter of 250 μm, having a porous sheath, of which the thickness is 2.7 μm and the average pore diameter, measured by SEM, is 585 nm).

The regression curves were constructed by measuring the electrochemical response of the wines (round and grey symbol in FIGS. 11, 12, 13), of the wines with dosed additions of SO₂ (diamond and black symbols in FIGS. 11, 12, 13) and of the wines with added ethanal (square and white symbol in FIGS. 11, 12, 13). The addition of ethanal makes it possible to combine the free SO₂ present in the wines and the recorded cyclic voltammogram corresponds in a way to the base line, the signal due to the free SO₂ being eliminated.

These curves highlight a linear increase of the current as a function of the concentration of free SO₂ for the three types of wine. The slopes of these curves are very close, but are on the other hand lower than that obtained in the case of the model solution of example 4, which reflects the effect of the wine matrix on the measurement of the concentration in free SO₂.

### Example 6: Measurement of the pH and of the Concentration in Molecular SO₂

A pH measurement was performed by following the previously described measurement protocol, starting from the position of the peak of reduction of the gold oxides described above and with an abacus established beforehand, linking the position of this peak to the pH value.

The position of the peak of reduction of the gold oxides varies with the pH (measured with a pH meter) as shown in FIG. 14.

The measurement of the pH can thus be obtained via the measurement electrode, without having to rely on an additional pH electrode in solution.

Thus, there is a linear relationship between the position of the peak of reduction of the gold oxides and the pH, in particular in the pH range of wines (pH 3 to 4), as shown in FIG. 15.

From the measurement of the concentration of free SO₂ obtained, of the pH, of the determination, in parallel of the temperature (T) of the sample using a conventional sensor, and of the alcohol titration (ABV) of the sample, it is possible to work back to the concentration of molecular or active SO₂ thanks to the two following formulae (see: Le SO₂ en Cenologie, Jacques Blouin, Dunod, 2014):

active SO₂(% free SO₂)=100/[10^{(pH-pK1)+}1]

with

pK₁=1.9499+(T−20)×0.0322+(ABV−10)×0.01971.

The temperature is measured on the sample just before, during or after the implementation of the dosage method of the invention. The alcohol titration is either determined by a measurement performed by the person implementing the method of the invention just before, during or after the implementation of the dosage method of the invention, or it is known because it has been measured beforehand, for example by the winemaker.

1. An electrochemical method for the detection and/or quantification of sulphur dioxide (SO₂) in its non-complexed forms called “free” in an aqueous or hydroalcoholic liquid-food, comprising:
a) the introduction in the liquid-food of a measurement electrode of which the active surface in contact with the liquid-food is entirely in gold,
b) the measurement of the variation of the current produced by the oxidisation of the free SO₂ present in the liquid-food, during potential scanning by cyclic voltammetry.

2. The method according to claim 1, wherein at step b), the potential scanning can be performed between +0.1 V and +1.9 V, using an Ag/AgCl reference electrode, at potential scanning speeds comprised between 1 mV·s⁻¹ and 10,000 mV·s⁻¹.

3. The method according to claim 1, wherein the liquid-food is wine.

4. The method according to claim 1, wherein the measurement electrode has a Rₜₕₑₒ ratio between its developed surface area (A_{dev}) and its apparent surface area (Aₐₚₚ) greater than or equal to 3.

5. The method according to claim 1, wherein the measurement electrode has a Rₜₕₑₒ ratio comprised between 3 and 100, preferably between 3 and 20.

6. The method according to claim 1, wherein it further comprises, prior to step b), a step b1) of potential scanning during a cyclic voltammetry, performed between +0.1 V and +1.5 V, using an Ag/AgCl reference electrode, at potential scanning speeds comprised between 1 mV·s⁻¹ and 10,000 mV·s⁻¹, without a measurement of the current variation.

7. The method according to claim 1, wherein it further comprises:
prior to step b), a step b2) of establishing an abacus providing the pH value of the liquid-food as a function of the position of the peak of reduction of the gold oxides during potential scans by cyclic voltammetry performed in said liquid-food at different pH values, using an Ag/AgCl reference electrode, at potential scanning speeds comprised between 1 mV·s⁻¹ and 1,000 mV·s⁻¹,
the determination of the alcohol titration, ABV, of the liquid-food, and
during step b) the measurement of the temperature T of the liquid-food, which provides the percentage of molecular SO₂ contained in the non-complexed SO₂, thanks to the following formula:
molecular SO₂%=100/[10^{(pH-pK1})+1]
wherein pK₁=1.949+(T−20)×0.0322+(ABV−10)×0.01971.

8. A method for regenerating in the liquid-food an electrode of which the active surface is entirely in gold, wherein it comprises:
a) the introduction in the liquid-food of an electrode, of which the active surface in contact with the liquid-food is in gold, and
b1) the implementation of cyclic voltammetry with potential scanning comprised between +0.1 V and +1.5 V, using an Ag/AgCl reference electrode, at potential scanning speeds comprised between 1 mV·s⁻¹ and 10,000 mV·s⁻¹, without a measurement of the current variation.

9. An electrochemical device for the detection and/or quantification of the free SO₂ in an aqueous or hydroalcoholic liquid-food, comprising:
a reference electrode
a counter-electrode
a measurement electrode, of which the active surface is entirely in gold, said measurement electrode having a Rₜₕₑₒ ratio between its developed surface area (A_{dev}) and its apparent surface area (Aₐₚₚ) greater than or equal to 3.

10. The device according to claim 9, wherein the measurement electrode is an electrode comprising a support covered with a porous sheath in gold, the sheath having a thickness and a pore size such that the measurement electrode has a Rₜₕₑₒ ratio between its developed surface area (A_{dev}) and its apparent surface area (Aₐₚₚ) greater than or equal to 3, preferably comprised between 3 and 100, and more preferably comprised between 3 and 20.

11. The device according to claim 9, wherein the measurement electrode is an electrode comprising a support covered with a rough layer of gold constituted of gold crystallites of nano to micrometric dimensions such that the measurement electrode has a Rₜₕₑₒ ratio between its developed surface area (A_{dev}) and its apparent surface area (Aₐₚₚ) greater than or equal to 3.
